(19) United States
(12) Patent Application Publication
(10) Pub. No.: US 2009/0149809 A1
(43) Pub. Date: Jun. 11, 2009

(54) OVERLAPPING DISPLAY

(76) Inventors: Markus Bollenbach, Bern (CH); Edgar Hommann, Grossaffoltern (CH); Benjamin Scherer, Uster (CH); Celine Kaenel-Jost, Oberburg (CH); Ursina Streit, Bern (CH); Daniel Kuenzli, Langendorf (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/191,097

(22) Filed: Aug. 13, 2008

Related U.S. Application Data
(63) Continuation of application No. PCT/CH2007/000063, filed on Feb. 7, 2007.

(30) Foreign Application Priority Data
Feb. 14, 2006 (DE) DE 102006006796.7

(51) Int. Cl. A61M 5/31

(52) U.S. Cl. 604/111

(57) ABSTRACT

A device for administering an injectable product including a housing, a viewing element formed on or in the housing to create a view area, an emptying display movable inside and/or relative to the housing at least partially in or through the view area, and a view release member movable relative to the housing, wherein the visiability of the emptying display can be changed, reduced or eliminated by moving the view release member.

### CROSS-REFERENCED RELATED APPLICATIONS

This application is a continuation of International Patent Application No. PCT/CH2007/000063 filed Feb. 7, 2007, which claims priority to German Patent Application No. DE 10 2006 006 796.7 filed Feb. 14, 2006, the entire contents of each of which are incorporated herein by reference.

### BACKGROUND

The present invention relates to devices for injecting, administering, dispensing, delivering or infusing a substance, and to methods of making and using such devices. More particularly, it relates to a device for administering an injectable product or substance, for example a liquid medicament such as insulin, heparin, a growth hormone, an osteoporosis preparation, etc. More particularly, in some embodiments, the device might be an injection device such as an injection pen or an automatic injector.

Devices for injecting, administering, dispensing, delivering or infusing a substance are known in many designs. In the case of such devices, especially automatic injectors, it may be of advantage for the user of the device to know the operational state of the device or what mode the device is in, for example, the piercing mode, administering mode, etc.

To this end, U.S. Pat. No. 6,692,469 proposes an injection device comprising a sleeve for accommodating a syringe and a trigger mechanism connected to the sleeve. The housing of the trigger mechanism has a window through which can be seen markings indicating whether the injection device has been triggered or not.

Published international patent application 2004/047891 discloses a device for automatically injecting a substance. The automatic injector described in the published document consists of two main components, namely a reservoir part accommodating a filled syringe and a power pack accommodating the parts used for automatically piercing and dispensing with the syringe. The power pack has a display window made from a transparent material, enabling a displaceably mounted display to be observed from the outside. In one embodiment of the device, the display is visible underneath the transparent sleeve in every operating mode. The display does not move until the product is being dispensed, once a needle attached to the syringe has effected a piercing action. Prior to the piercing and dispensing operation, a trigger knob can be pulled back so that a sleeve of a warning color can also be seen through the transparent sleeve, from which the user knows that the device is ready for triggering. In another embodiment, the dispensing display already moves as the needle effects the piercing action, and the display is covered by a cover from the start to the end of the piercing operation inclusively. The dispensing display can not be seen until the dispensing operation starts. The dispensing display protrudes from the non-displaceable cover. In either of the embodiments, a user of the device is not able to tell from the display whether the device is still in piercing mode or not.

### SUMMARY

An object of the present invention is to provide a device and method for administering an injectable product which offers a user of the device an easy way of seeing the different modes of the device.

In some embodiments, the device for administering an injectable product in accordance with the present invention may be an injection device, e.g. an automatic injector or injection pen. In such devices, especially in automatic injectors, administering the injectable product may involve several sequences. For example, there might be a piercing sequence during which a needle pierces the body tissue and a dispensing sequence during which the liquid medicament is dispensed. The dispensing sequence and/or the piercing sequence, which can then be followed by a dispensing sequence, is advantageously automatically activated or triggered.

In some embodiments, an injection device in accordance with the present invention is generally elongated, slender and cylindrical, having a housing, which may comprise two or more housing parts which can be or are connected to one another detachably or non-detachably. Screw fitting, bayonet fitting, catch fitting or other suitable connecting structures may be used for this purpose. A mechanical sequence controller which controls the piercing and/or dispensing sequence is accommodated in a proximal housing part and a product container is accommodated in a distal housing part. (As used herein, “proximal” means the end lying opposite the needle or the rear end, and “distal” means the end at which the needle is disposed or the front end). The product container, e.g. an ampoule, can be replaceably inserted into the distal housing part or may be formed directly by the distal housing part. In some embodiments, the housing may be a one-piece design.

In some embodiments, the present invention compries a device for administering an injectable product comprising a housing, a viewing element formed on or in the housing to create a view area, an emptying display movable inside and/or relative to the housing at least partially in, through or past the view area, and a view release member movable relative to the housing, wherein the visiability of the emptying display can be affected, changed, reduced or eliminated by moving the view release member.

In some embodiments, the present inventon comprises a device for administering an injectable product, comprising: a housing with a housing wall, a viewing element which is formed on the housing and with which a view area can be observed, an emptying display which is movable inside the housing and relative to the housing, at least partially through the view area, and a view release member which is movable relative to the housing, wherein the view of the emptying display can be faded over and/or freed with the view release member.

In some embodiments, the housing has a housing wall. A viewing or look-through element, window or port is provided on the housing, in the housing wall. The look-through element enables a user of the device to see through the housing wall into the interior of the housing from outside the wall. This region may be thought of and referred herein as the viewing region or area. In particular, the viewing region may be a projection of the look-through element in the radial direction with reference to the longitudinal axis of the device. “Disposed in the viewing region” or “in the view area” refer to elements which are within the projection of the look-through element but which are not visible through the look-through element, for example because they are covered by another part. In some embodiments, the look-through element is a window or an orifice in the housing wall. In some embodiments, the look-through element is a part made from a transparent material incorporated into the housing wall or inserted in the wall. In some preferred embodiments, the transparent material is glass or a clear or colorless plastic. In some preferred embodiments, the look-through element has a magnifying or reducing optical effect. To this end, the look-through element may be thought of, referred to or take the form of a lens or the like.

In some embodiments of the device in accordance with the present invention, the device has a mechanical dispensing display, which can be moved at least longitudinally inside and/or relative to the housing at least partially through or past the viewing region. At least some regions of dispensing display can therefore be seen by the user depending on the operational mode or state in or to which the device has been switched or moved. The purpose of the dispensing display is to provide the user with information about whether the product or substance to be administered was dispensed and/or how much of the product was dispensed.

In some embodiments, the product is contained in a product container, which is contained in the device or can be introduced into the device. The product is dispensed from the product container by a plunger acting on the product. When the product is being dispensed, the plunger moves relative to the product container. The plunger and dispensing display are coupled with one another so that when one of the dispensing display or plunger moves, the other at least partially also effects the same movement. For example, one of them can travel a specific distance during the movement while the other travels a distance that is longer, shorter or of equal length. For example, one may move at a specific speed which is higher or lower or equal to the speed of the other one. In some preferred embodiments, one effects the movement in the same direction as the other. In some preferred embodiments, the plunger moves a longer distance than the dispensing display during the dispensing movement. The plunger travels a part of the distance it must cover for full dispensing in such a way that the movement of the plunger is completely uncoupled from the dispensing display across this part-distance. As the plunger is moving across this part-distance, no force acting on the dispensing display varies, for example. In some preferred embodiments, no force acts on the dispensing display while the plunger is moving across the part-distance, and/or the dispensing display is stationary relative to the housing as the plunger moves across the part-distance.

In some preferred embodiments, when the plunger has travelled the part-distance in which its movement is uncoupled from the dispensing display, the plunger travels a remaining distance for dispensing the product, across which the movement of the plunger is coupled with the dispensing display. The coupling may be of the type whereby the dispensing display effects the same movement as the plunger. In some preferred embodiments, the dispensing display travels the same distance as the plunger across the remaining distance, and/or at the same speed as the plunger.

Depending on the position of the plunger, the dispensing display or a portion thereof is disposed with a specific area in the viewing region and/or is visible. Several areas may be provided on the dispensing display. If there are several areas, these may be disposed one after the other in the direction of movement so that different areas pass through the viewing region during the movement through the viewing region. In some preferred embodiments, every area contains and/or provides different information from the other areas or an adjacent area. Accordingly, the user of the device is able to tell from the information which appears under or in the look-through display at a specific instant if or how much product has been dispensed from the product container.

For example, in some embodiments, a first area disposed in the viewing region is moved out of the viewing region during dispensing of the product so that another area is disposed in the viewing region when the product has been fully dispensed. In some embodiments, an intermediate area may be provided between the first area and second area, which is disposed in the viewing region or moves through the viewing and can be seen as the product is being dispensed. As a result, the user of the device is able to tell whether dispensing of the product has started, but has not yet fully terminated (termination would be readily apparent when the second area is disposed in the viewing region).

In some embodiments, the information provided in the area or areas may take the form of a material property, a character, an image, a color and/or a color code, and at least the information of the first area is different from that of the second area. The information of the first area may be of the same general type, such as different letters. The information may also be of different types, in which case the first area may contain a letter and the second area a color code. The material property may be the nature of the material used, for example, or a surface structure. A character may be a letter, a symbol, a number, etc. A color code might be a color that is relevant to the respective dispensing mode. For example, the first area may be of a less noticeable color, such as “green,” and the second area may be of a more noticeable color or a signalling color such as “red.” The user of the device will initially see the less noticeable color through the look-through element. As dispensing of the product progresses, the user of the device will see the area with the signalling color through the look-through element as it moves into the viewing region.

In some embodiments, to provide a user of the device with information indicating that the device is still in the product dispensing sequence, an intermediate area may be provided between the first area and the second area which may contain both information from the first area and information from the second area. Alternatively, the intermediate area may contain separate information which might be different from, of a different type from, or of the same type as the information of the first and/or second area. The intermediate area may contain a predominant color code of the adjacent area more or less at the point at which it adjoins the adjacent area. Since the information of the first and second area is different, the color code of the first area may change to the color code of the second area during travel to the second area. The change in color code may be homogeneous for example, so that a plurality of colors which are perceptible to the human eye are disposed in the intermediate area or the colors are mixed. The change in color code may also be non-homogeneous, in which case blocks, for example two blocks, of different colors are provided, the respective width proportions or surface proportions of which become larger or smaller. The intermediate area may incorporate the colors of the first and second area. For example, the proportions of color of the first area during travel from the first to the second area may decrease and the proportions of color of the second area may increase. In some embodiments, the color of the second area increases during travel from the first area to the second area to the same degree as the color of the first area decreases during travel from the first area to the second area.

In some preferred embodiments, the device also has a view-releasing element, by which the view onto the dispensing display can be screened out. “Screening out” means that the view onto the dispensing display can be affected, changed, blocked, reduced or eliminated so that the dispensing display is not visible or the view onto the dispensing display is made possible via a transparent material, although in this case, the part disposed behind the view-releasing element is screened out by specific information of the view-releasing element. The information might be based on a specific color of the transparent material or information based on characters or a letter or an image applied to the transparent or opaque view-releasing element.

In some embodiments, the view-releasing element is able to move relative to the housing, thereby exposing the view onto or into the dispensing display. “Exposing the view onto the dispensing display” means that the dispensing display can be seen or that when screened with a transparent material, the information which the user of the device can see when screened then changes on exposure. In some preferred embodiments, the view-releasing element moves in the longitudinal direction, and the view onto the dispensing display is exposed as the view-releasing element moves in the distal direction. Like the dispensing display, the view-releasing element is able to move at least partially through or past the viewing region. The view-releasing element may be disposed outside the housing for example, so that it can be pushed over the look-through element or, in some preferred embodiments, the view-releasing element may be disposed under the look-through element or inside the housing in the viewing direction. In some preferred embodiments, the view-releasing element is disposed between the housing wall and dispensing display, in which case the view of the dispensing display is advantageously screened or exposed.

In some embodiments, the product container contained in the device or which can be introduced into the device may be connected to or carry a needle, which is able to pierce a patient's skin. The needle can be displaced relative to the housing along its longitudinal axis. The needle is enclosed by the housing or by a needle guard sleeve attached to the housing so that the needle is able to project or extend out from the housing or the needle guard sleeve distally for the piercing operation, for example by a certain piercing length. In some embodiments, the needle is coupled with the view-releasing element so that when one of the view-releasing element or needle moves, the other effects at least some of the same movement as the other one. In some embodiments, the view-releasing element moves in the same direction as and/or by approximately the same distance and/or at the same speed as the needle during piercing. In some embodiments, the needle may be attached to the container directly so that it can move relative to the container or is axially fixed to the container, so that the needle and container are able to effect a joint piercing movement. The designs used for coupling the needle with the view-releasing element may also be used for coupling the view-releasing element with the container.

In some embodiments, the needle and/or the product container can be moved along the longitudinal axis relative to the housing between an initial position and a piercing position. In the initial position, the device is in the “non-operating mode,” in other words is in a state prior to triggering the dispensing and piercing sequence, wherein the needle is disposed inside the housing or needle guard sleeve. In the piercing position, the needle extends distally out of the housing or needle guard sleeve by a desired piercing length. The needle/product container is moved into the piercing position by triggering the piercing sequence in the initial position.

In some embodiments, the view-releasing element has a first area disposed in the viewing region when the product container is in its initial position or the needle is in its initial position. For example, the first area contains information which can be seen through the look-through element. From this information, the user of the device has a visual way of telling that the needle has not yet pierced or the device is in its initial position. in some embodiments, the information of the first area is a material property of the material of the first area for example, a character, an image and/or a color code. The information may be similar to the information of the first or second area of the dispensing display. In some preferred embodiments, the first area is transparent or opaque. If the first area is opaque, it is not possible to see behind the view-releasing element in the viewing direction. If the view-releasing element has a transparent first area, it is possible to see behind the view-releasing element in the viewing direction. In some embodiments, the first area may be clear or colorless or may be provided with a transparency of a specific color. In embodiments in which the first area is transparent, further information may be provided on the surface of the first area.

In some preferred embodiments, a second area is disposed adjacent to the first area which is disposed in the viewing region when the needle or product container is disposed in the piercing position. The advantage of this is that the user of the device has a way of visually checking when the needle has been extracted or extended from the housing or needle guard sleeve by the desired piercing length or has pierced the body tissue. The user can read the dispensing display through the look-through element and the second area of the view-releasing element. The second area of the view-releasing element may be smaller than, bigger than or approximately the same size as the look-through element, and it may be of approximately the same shape as the look-through element.

In some embodiments, the second area adjoins the view-releasing element or is a window in the view-releasing element. The second area may be, but is not necessarily, an integral part of the view-releasing element. For example, the view-releasing element may terminate proximally of the first area so that when the needle or container moves out of the initial position into the piercing position, the view-releasing element or first area is moved essentially completely out of the viewing region. The area which proximally adjoins the view-releasing element may therefore be thought of and/or termed a second area because in functional terms, the view of the dispensing operation is specifically and generally exposed in the second area.

In another embodiment, the second area is an element of the view-releasing element. This being the case, the second area is a window made from a transparent material. In some preferred embodiments, if the second area is transparent, the material clear or colorless and transparent. If a first area is transparent, the transparent material of the second area is of a different color from the transparent material of the first area. This provides the user of the device with an easy and reliable way of telling when the needle is in its piercing position.

In another embodiment, an intermediate region is provided in the longitudinal direction of the device between the first area and the second area, which moves at least partially through the viewing region or is disposed in the viewing region when the product container or the needle moves from the initial position into the piercing position. The intermediate area may comprise a character, color, an image, a color code and/or material property, by which it is possible to tell that the product container and/or needle are moving. In some preferred embodiments, the features of the intermediate area of the view-releasing element have the same features as the intermediate area of the dispensing display.

In one embodiment, the dispensing display in the initial portion can be disposed in the viewing region, with its first area in the viewing region, in which case the dispensing display or its first area is screened by the view-releasing element, by the first area thereof. In this embodiment, the view-releasing element moves relative to the housing and the dispensing display during the piercing sequence. In other words, in the initial position, the first area of the view-releasing element and the first area of the dispensing display are disposed in the viewing region.

In another preferred embodiment, the first area of the dispensing display is disposed outside of the viewing region in the initial position. For example, the first area of the dispensing display is disposed in the region of the second area of the view-releasing element at the same axial height or position. In the initial position, the first area of the view-releasing element is disposed in the viewing region and can be seen. When the piercing sequence is triggered, the view-releasing element and the dispensing display are pushed together in the distal direction relative to the housing. The view-releasing element and the dispensing display effect the piercing movement of the needle together, namely without any relative movement. In the piercing position and prior to dispensing the product, the first area of the dispensing display and the second area of the view-releasing element are disposed in the viewing region, so that the user of the device can see the first area of the dispensing display and read its information. Consequently, the user of the device is able to tell visually that the needle is in its piercing position and no product has yet been dispensed.

In some embodiments, once the piercing position is reached, the dispensing sequence is automatically or manually initiated. The view-releasing element does not effect any movement relative to the housing or remains stationary relative to the housing during the product dispensing sequence. During the product dispensing sequence, the dispensing display moves relative to the housing and to the view-releasing element. This being the case, the first area of the dispensing display is moved away from the viewing region so that the user can initially read the information of the intermediate area of the dispensing display, if any is provided, and then the information of the second area of the dispensing display.

In some embodiments, the intermediate area and/or the second area of the dispensing display are omitted so that the user is able to see behind the dispensing display in the viewing direction during or after dispensing of the product. However, in some preferred embodiments, the second area of the dispensing display is disposed in the viewing region once the product has been dispensed and can be read at a glance through the look-through element and the second area of the view-releasing element.

In some preferred embodiments, an elastic means, for example a forward driving spring in the form of a coil spring, is provided which is able to move the view-releasing means, the product container, the needle and/or the dispensing display in the piercing direction. When the device is in the initial state, the elastic means is in a pre-tensed state and be blocked or locked to prevent it from relaxing. For example, the pre-tensed elastic means is supported on an element secured to the housing, e.g. a mechanism holder, and an element coupled with the view-releasing element or directly on the view-releasing element. An element coupled with the view-releasing element is a forward feed element, which drives the view-releasing element with it in the distal direction as the elastic means relaxes. The forward feed element is able to move along the longitudinal axis of the device relative to the housing. For example, the forward feed element may be connected to a lock element which is axially fixedly connected to the housing or mechanism holder and radially displaceable, to prevent a movement in the longitudinal direction. To this end, the lock element locate, lodges or locks in, with or to the forward feed element.

In some embodiments, a device in accordance with the present invention has an output element which can be moved along the longitudinal axis of the device and relative to the housing. The output element is able to act indirectly or directly on the plunger of the product container, via a plunger rod, for example and, the plunger rod may constitute the plunger. It would also be conceivable for the output element to assume the function of the plunger rod and act directly on the plunger. For example, the output element may constitute the plunger rod and plunger. In the initial position and during the piercing sequence, the output element is locked to prevent a movement in the longitudinal direction relative to the forward feed element. For example, the forward feed element has a lock element which is able to locate or couple in, to or with the output element. The lock element is axially stationary and radially displaceable relative to the forward feed element. For example, the output element has a collar projecting radially outwardly, behind which the lock element locates or latches, thereby preventing a movement of the output element in the distal direction. In some preferred embodiments, the output element is sleeve-shaped, and an elastic means, e.g., a dispensing spring in the form of a coil spring, is provided to drive the output element forward (i.e., distally) in the dispensing direction during the product dispensing sequence. In some embodiments, the elastic means may be accommodated inside the output element.

In some preferred embodiments, an injection device in accordance with the present invention comprises a trigger element which pushes the lock element out of engagement with the forward feed element so that the forward driving spring can relax. In one preferred embodiment, the forward driving spring drives the forward feed element with it, which in turn drives the dispensing display and the view-releasing element with it. (In some embodiments, the forward feed element and view-releasing element may be integral.) The forward feed element also drives the product container or the needle with it. The dispensing display and view-releasing element are driven by the forward feed element by the same distance and/or at the same speed, without any relative movement with respect to one another. During the piercing sequence, the output element is also be driven by the forward feed element without any relative movement with respect to the forward feed element.

When the piercing sequence has terminated, the output element can be moved relative to the forward feed element and the forward feed element is blocked or locked to prevent a movement relative to the housing. The lock element, which locks the output element in the initial position and during the piercing sequence is provided as a releasing the locked engagement with the output element at the end of the piercing sequence or at the start of the product dispensing sequence, simultaneously moves into a locked engagement with an element fixedly disposed on the housing, for example the mechanism holder. The distance which the forward feed element travels during the piercing sequence essentially corresponds to the distance which the needle and/or the product container travel during the piercing sequence.

Once the output element has been released at the start of the product dispensing sequence or at the end of the piercing sequence, the output element is able to move by a part-distance without driving the dispensing display with it, and the plunger moves by its part-distance. After the part-distance of free movement, the output element then drives the dispensing display in its movement in the dispensing direction. At the same time, the output element pushes the plunger of the container by the same amount, for example by the remaining distance, as the dispensing display. The second area of the dispensing display is then disposed exactly in the viewing region and can be seen when the plunger has been pushed completely into its most distal position in the container. The advantage is that a user can tell visually through the look-through element that the plunger has now reached its most distal position and the product has been completely dispensed.

In a preferred embodiment, the product container may be an element of a module which can be inserted in or introduced into the device. The module comprises a guide element which can be connected axially and fixedly to the distal housing part, and an ampoule container which can be moved lengthways relative to the guide element and in which the product container is or can be accommodated. The ampoule holder is pushed by a spring into a proximal position, namely the initial position. During the piercing sequence, the ampoule holder and product container are pushed together against the spring force into a distal position, namely the setting position. The guide element has at least one catch means so that the ampoule holder and guide element can be latched in the piercing position so as to be axially stationary. The module is driven with the product, the plunger closing off the product and a plunger rod attached to the plunger. An appropriate needle with a needle guard may also be fitted so that the needle guard merely has to be removed from the module in readiness for use.

A method in accordance with the present invention comprises displaying different operational modes of injection device for administering an injectable product. As a result of the method, various items of information are displayed, which can be read through a viewing or look-through element associated with the device. These items of information comprise at least information to the effect that the product container disposed in the device is in an initial position, to the effect that the product container is in a piercing position and to the effect that the product contained in the container has been dispensed.

### BRIEF DESCRIPTION OF THE DRAWINGS

FIG. 1A is a sectional diagram illustrating an embodiment of an injection device in accordance with the present invention in an initial state,

FIG. 1B is a sectional diagram rotated about the longitudinal axis by 90° showing the injection device illustrated in FIG. 1A,

FIG. 2A is a sectional diagram showing the injection device illustrated in FIG. 1A in a mode positioned on body tissue,

FIG. 2B is a sectional diagram rotated about the longitudinal axis by 90° showing the injection device illustrated in FIG. 2A,

FIG. 3A is a sectional diagram showing the injection device illustrated in FIG. 1A in a triggered mode in the initial position at the start of a piercing sequence,

FIG. 3B is a sectional diagram rotated about the longitudinal axis by 90° showing the injection device illustrated in FIG. 3A,

FIG. 4A is a sectional diagram showing the injection device illustrated in FIG. 1A in a piercing position at the end of the piercing sequence and at the start of a product dispensing sequence,

FIG. 4B is a sectional diagram rotated about the longitudinal axis by 90° showing the injection device illustrated in FIG. 4A,

FIG. 5A is a sectional diagram showing the injection device illustrated in FIG. 1A in a post-dispensing state at the end of the product dispensing sequence,

FIG. 5B is a sectional diagram rotated about the longitudinal axis by 90° showing the injection device illustrated in FIG. 5A,

FIG. 6A is a sectional diagram showing the injection device illustrated in FIG. 1A in a locked mode,

FIG. 6B is a sectional diagram rotated about the longitudinal axis by 90° showing the injection device illustrated in FIG. 6A,

FIG. 7 is a schematic diagram of an embodiment of an individual view-releasing element; and

FIG. 8 is a schematic diagram of an embodiment of an individual dispensing display.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making the invention and/or its components may be selected from appropriate materials such as metal, metallic alloys, ceramics, plastics, etc.

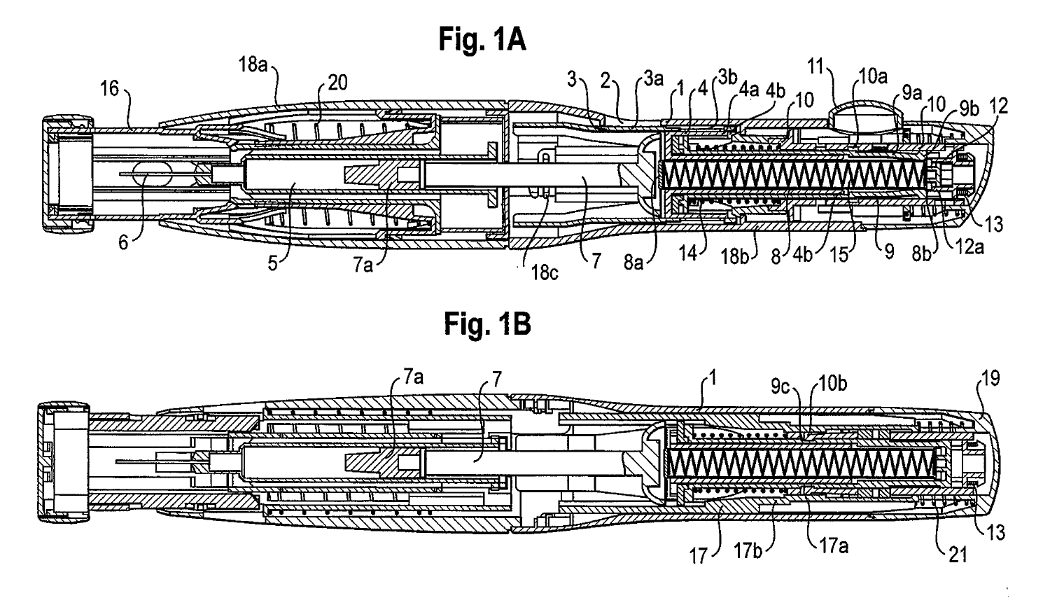

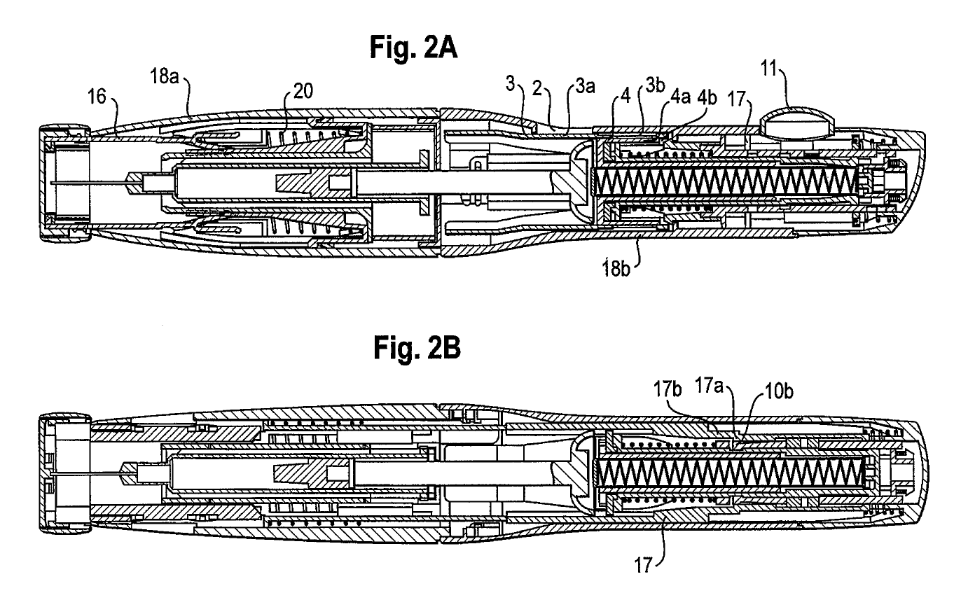

FIGS. 1A to 6B illustrate an embodiment of an injection device in accordance with the present invention in the form of an automatic injector. The Figures show the device in differently switched states and from different aspects. The parts of the device will be described below with reference to FIGS. 1A and 1B and this description likewise applies to FIGS. 2A to 6B. The injection device has a mechanism with a mechanical sequence controller, by which a product container 5 contained in the injection device is pushed in the distal direction so that the needle 6 attached to the product container 5 pierces a body tissue of a patient and, after the piercing sequence, the plunger 7a is moved in the product container 5 in the distal direction during a dispensing sequence so that the product contained in the container 5 is dispensed through the needle 6 into the body or body tissue of a patient.

The injection device has a distal housing part 18a and a proximal housing part 18b, which are connected by a releasable connection, e.g. a bayonet fitting 18c. Disposed in the proximal housing part 18b is a mechanism holder 10, which is connected to the proximal housing part 18b so that it is prevented from rotating and moving axially. To secure the mechanism holder 10 so that it is axially secured, it is axially clamped by its end faces between a projection of the proximal housing part 18b extending radially inwards and a cap 19 fitted proximally on the proximal housing part 18b. The mechanism holder 10 extends around a forward feed element 9, which can be displaced relative to the mechanism holder 10 along the longitudinal axis of the injection device. The sleeve-shaped forward feed element 9 is able to slide by its external circumferential face along the inner circumferential face of the sleeve-shaped mechanism holder 10. Disposed between a distal end face of the mechanism holder 10 and a proximal end face of an annular flange disposed at the distal end of the forward feed element 9 is a spring 14, e.g. a coil spring which, when placed under pressure in a pre-tensed state, biases or urges the forward feed element 9 in the distal direction relative to the mechanism holder 10 by a forward biasing force.

The distal end face of the annular flange disposed distally on the forward feed element 9 is able to act in a direction along the longitudinal axis on a view-releasing element 3 which is displaceable along the longitudinal axis in the proximal housing part 18b. The view-releasing element 3 is coupled with the product container 5 so that when the view-releasing element 3 moves in the distal direction, the product container 5 is likewise moved in the distal direction, namely in the piercing direction of the needle 6. The movement of the view-releasing element 3 in the distal direction does not yet cause the plunger 7a to move inside and relative to the product container 5. A spring 20 pre-tensed under pressure forces the product container 5 and the view-releasing element 3 in the proximal direction of the injection device. The force of the spring 20 by which the view-releasing element 3 is forced in the proximal direction is weaker than the force of the spring 14 by which the forward feed element 9 and the view-releasing element 3 can be moved in the distal direction when the forward feed element 9 is released, enabling it to move along the longitudinal axis so that the product container 5 with the needle 6 can be moved in the distal direction to enable the needle 6 to pierce the body tissue.

Disposed inside the forward feed element 9 is a sleeve-shaped output element 8, which is able to move relative to the forward feed element 9 along the longitudinal axis for a dispensing sequence, once it has been released so that it can move axially. Disposed at the proximal end of the output element 8 is a collar 8b projecting radially outwardly. Disposed between the output element 8 and the forward feed element 9 is an annular groove, the groove width of which is wider than the amount by which the collar 8b projects out radially from the output element 8. To form the annular groove, provide a mount for the output element 8 and move the dispensing display 4, the dispensing display 4 extends with a sleeve-shaped portion from the distal end into the internal diameter of the forward feed element 9 and locates around the output element 8.

The distal end of the output element 8 has an end face 8a acting as a stop surface, on which a spring 15, e.g. a coil spring, can be supported. At its other end, the spring 15 pre-tensed under pressure is supported on a distal face of a stop element formed on a proximal region of the forward feed element 9. The spring 15, which may have a higher spring constant than the spring 14, is able to drive the output element 8 when it is released, in the distal direction along the longitudinal axis. The distal end face of the output element 8 moves into contact with a plunger rod 7 or with a dome-shaped element disposed at the proximal end of the plunger rod 7, causing the plunger 7a disposed distally on the plunger rod 7 to move inside the product container, as a result of which the product is dispensed through the needle 6 when the output element 8 is moved in the distal direction.

A lock element 12 is disposed on the proximal end of the forward feed element 9. The lock element 12 is able to move along the longitudinal axis relative to the forward feed element 9. The lock element 12 is forked and extends around the longitudinal axis with two lock portions 12A. The lock portions are respectively guided in a groove-shaped guide formed by the forward feed element 9. The lock element 12 is biased by a force in the distal direction by a spring 13. The spring 13 is supported on the proximal end of the lock element 12 and on an element disposed proximally to it and fixedly relative to the forward feed element, namely on a retaining web. The spring 13 may be a coiled spring, a combined coil and spiral-shaped spring, or a leaf spring. In some embodiments, the retaining web may be made from a resilient material, for example, and may be of a shape which is able to bias the lock element 12 directly with a resilient force, in which case the spring 13 can be dispensed with because the lock element 12 assumes the function of the spring 13.

A movement of the lock element in the distal direction is prevented due to the lock element 12 being latched or blocked at its lock portions 12A by the collar 8b of the output element 8. The collar 8b is in turn prevented from moving in the distal direction by the cam-shaped lock elements 9b which are respectively disposed on an arm 9b and resiliently joined to the forward feed element 9, e.g. integrally.

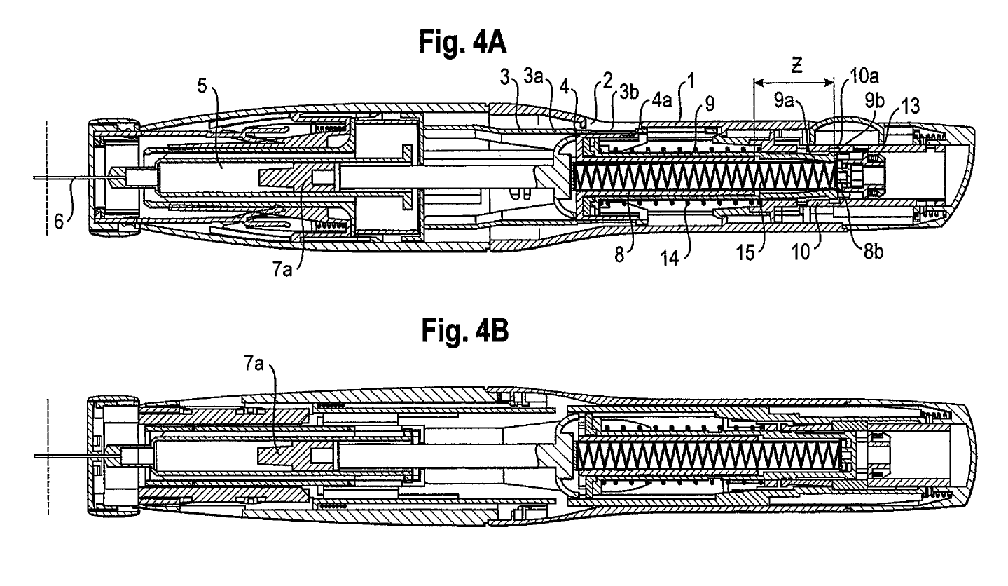

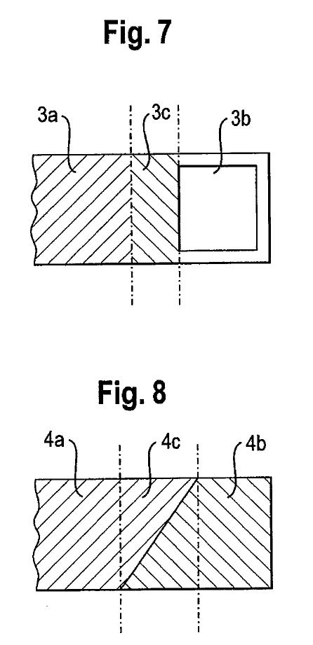

The housing wall 1 of the proximal housing part 18b has a look-through or viewing element 2, in the form of a window. Through the look-through element 2, the observer can see in the direction toward the inside of the device from the outside of the device. In FIGS. 1A to 3B, the view into the interior of the device is blocked by the view-releasing element 3. The view-releasing element 3 has a first area 3a, which is disposed at the same height as the look-through element 2 in the longitudinal direction of the device when the device is in the initial position, namely prior to the piercing and dispensing sequence. As also illustrated in FIG. 7, the first area 3a contains first information, indicated in the form of a hatched surface. The information could likewise be provided in the form of a specific color, for example red, or with the help of a character or letter (not illustrated). As a result of thes information of the first area 3a, the user of the device can tell that no product has yet been dispensed or no piercing operation has taken place. When, as illustrated in FIGS. 4A and 4B, the released forward feed element 9 is moved in the distal direction, the lock element 9b moves to approximately the same axial height as a catch groove 10a after travelling a specific distance which approximately corresponds to the distance by which the product container 5 is pushed for piercing purposes. The catch groove 10a is formed by the mechanism holder 10. The catch groove 10a for the lock elements 9b may be a respective recess or a recess extending in a circle. As illustrated in FIG. 4A, as soon as the catch groove 10a and the lock element 9b are at the same axial height, the spring force of the arms 9a, the spring force of the spring 13 and/or the spring force of the spring 15 in conjunction with the sliding of the collar 8b radially outwardly causes a movement into the catch groove 10a. The dispensing sequence is initiated as a result.

During the piercing sequence, the output element 8 with its pre-tensed driving spring 15 on the one hand, and the view-releasing element 3 and dispensing display 4 on the other hand are slaved or moved, driven by the spring 14. The view-releasing element 3 in turn drives the product container 5 together with the needle 6 attached to its distal end to effect a piercing movement. As this happens, the first area 3a of the view-releasing element 3 is moved out of the viewing region of the look-through element 2, as a result of which an intermediate area 3c of the view-releasing element 3, such as is illustrated in FIG. 7 for example, initially moves into the viewing region of the look-through element 2, from which the user of the device is able to tell that the needle 6 is performing the piercing sequence.

When the piercing position is reached, the second area 3b of the view-releasing element 3, which was initially covered by the housing wall 1, moves into the viewing region of the look-through element 2. The second area is provided in the form of a window. Since the dispensing display 4 is moved in the distal direction together with the view-releasing element 3 during the piercing sequence, the user of the device can look through the look-through element 2 and the second area 3b of the view-releasing element 3 to the dispensing display 4. Prior to dispensing product, the dispensing display 4 is disposed with a first area 4a at the same axial height as the second area 3b of the view-releasing element 3. At the end of the piercing sequence or the start of the dispensing sequence, the user is therefore able to read the information of the first area 4a of the dispensing display 4. As illustrated in FIG. 8, the first area 4a may contain information such as indicated herein by a hatched surface or in particular of a specific color, for example “yellow”. Due to the change in color which appears in the look-through element 2, the user knows that the operating mode of the device has changed.

As regards the dispensing display 4, instead of the design illustrated in FIGS. 1A to 6B, the dispensing display 4 may be extended in the distal and/or proximal direction. If the dispensing display 4 has a proximal extension, the second area 4b is disposed at or on the proximal extension. If the dispensing display 4 has a distal extension, the first area 4a is disposed at or on the distal extension.

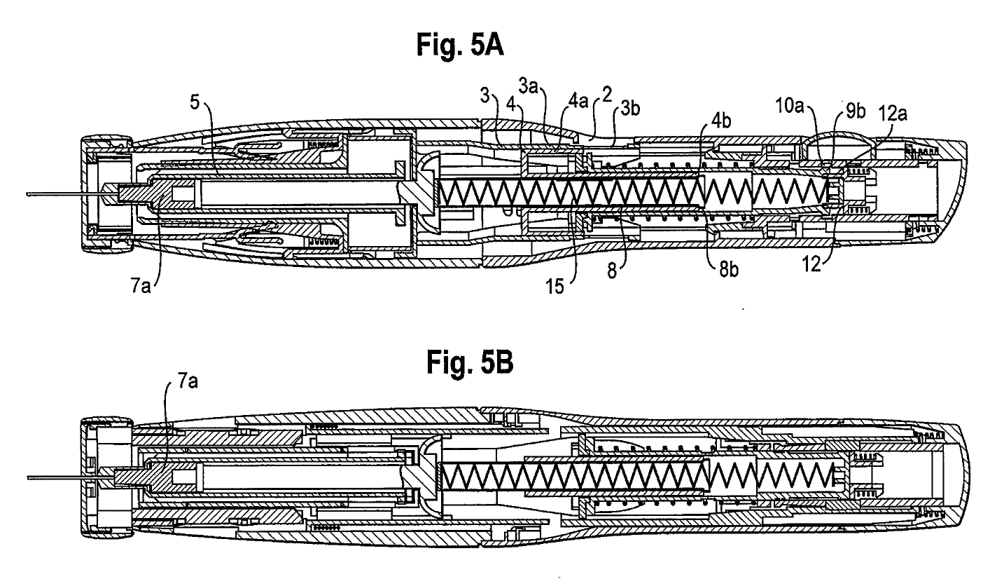

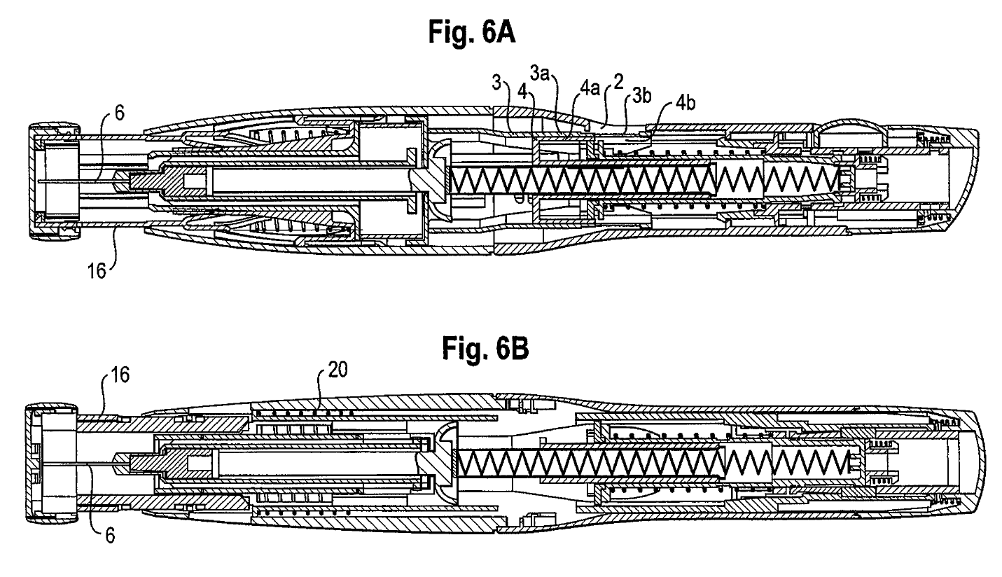

Once the dispensing sequence has started, the collar 8b is no longer locked by the lock element 9b, as a result of which the spring 15 drives the output element 8 in the distal direction and, thus, the plunger 7a via the plunger rod 7 to dispense product. At the same time, the lock element 12 is released for an axial movement so that the spring 13 of the lock element 12 pushes so far that the lock portions 12a push in front of the lock element 9b. The lock element 9b is prevented from moving out of the catch groove 10a because the lock portions 12a block the movement which the lock element 9b would have to effect to move out of the catch groove 10a. Consequently, the forward feed element 9 is joined to the housing so as to be axially stationary. As the output element 8 moves by the distance z, the output element 8 initially drives only the plunger rod 7 with it, which likewise moves the plunger 7a by approximately the distance z. The dispensing display 4 is not moved during travel across the distance z. After travelling the distance z, the radially projecting collar 8b of the output element 8 arrives in axial contact with a stop 4b disposed proximally of the sleeve-shaped portion of the dispensing display 4. Due to the driving force of the spring 15 and the collar 8b, the dispensing display 4 is also driven across the remaining distance making up the total stroke of the plunger less approximately the distance z, until the plunger 7a has completely forced the product out of the product container 5. During the product dispensing sequence, the dispensing display 4, particularly its first area 4a, is moved out of the viewing region. Since the dispensing display 4 does not have a second area 4b but rather the second area 4b adjoins the dispensing display 4 proximally, the information to the effect that product has been dispensed is displayed by releasing the view underneath the dispensing display 4, as illustrated in FIGS. 5A and 6A.

Alternatively, in the case of an injection device with a dispensing display 4 based on the design illustrated in FIG. 8, the first area 4a of the dispensing display 4 is moved in the distal direction out of the viewing region of the look-through element 2 during the product dispensing sequence. This being the case, an intermediate area 4c is initially moved into the viewing region, from which the user of the device can tell that the product dispensing sequence has started, but has not yet ended. When the plunger reaches the end position, the area 4b is moved into the viewing region of the look-through element 2, from which the user knows that the product dispensing operation has terminated.

In the case of FIG. 8, the information contained in area 4b is symbolically indicated by a hatched surface. For example, the area 4b may have a “green” surface. The intermediate area 4c contains both the information of the first area 4a and the information of the second area 4b. During the progressive movement from the first area 4a to the second area 4b in the intermediate area, the information of the first area 4a becomes smaller and that of the second area 4b larger. Therefore, the user of the device can estimate how far the product dispensing operation has progressed on the basis of the degree to which the intermediate area 4c has already moved in the longitudinal direction.

In some preferred embodiments, the dispensing movement of the output element 8 does not take place until the needle 6 has fully completed the piercing movement, and/or the piercing sequence can not be initiated until a transmitting element 16 in the form of a needle cover has been pressed sufficiently firmly onto the body tissue at the piercing site. As the device is pushed onto the body tissue, the transmitting element 16 is pushed relative to the distal housing part 18a. The proximal housing part 18b provides a mount for a switch element 17 so that it can be moved along the longitudinal axis. As the transmitting element 16 is moved in the proximal direction, the switch element 17 is moved with it. The transmitting element 16 is pushed against the force of a spring 20 and/or a spring 21 in the proximal direction. The switch element 17 can be moved against the force of the spring 21 in the proximal direction.

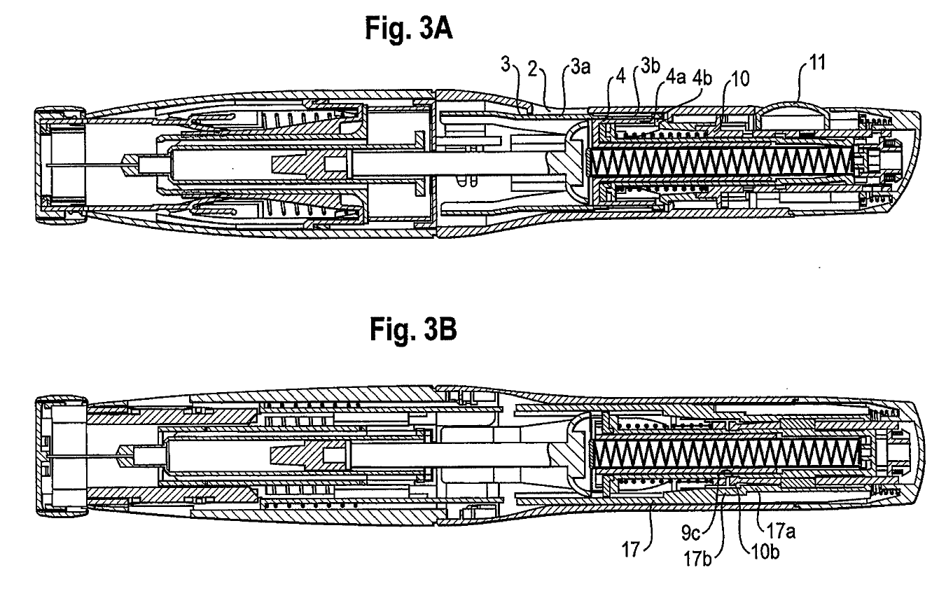

As illustrated in FIGS. 2A and 2B, when the switch element 17 has been moved in the proximal direction, the switch element 17 can be moved still farther in the proximal direction by a gear surface (not illustrated) disposed on the switch element 17 due to the movement of a trigger element 11 in the radial direction, so that, as illustrated in FIGS. 3A and 3B, the lock element 10b is no longer blocked on a retaining step 17a of the switch element 17. In this switched mode, the lock element 10b is disposed at the same axial height as a releasing step 17b which enables the lock element 10b to move out of the locked engagement with the forward feed element 9, thereby initiating the piercing sequence. The forward feed element 9 may have one or more recesses, which might be circular for example, in which the lock element 10b is able to engage to establish the locked engagement with the forward feed element 9.

As illustrated in FIGS. 6A and 6B, when the injection device is removed after the piercing and dispensing sequences have taken place, the needle guard 16 is pushed by a return spring 21 so far in the distal direction beyond the needle 6 that the needle 6 is covered or disappears completely into the device. This reduces any risk of injury to the user of the device. At the same time, once the trigger element 11 has moved back into its initial position and released the switch element 17, the switch element 17 is moved back into its initial position as illustrated in FIGS. 1A and 1B, for example. By contrast with the initial position (FIG. 1A for example), the user can tell from the changed information underneath the look-through element 2 that the device has already been used.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering an injectable product comprising a housing, a viewing element associated with the housing to create a view area, an emptying display movable inside and relative to the housing at least partially in or through the view area, and a view release member movable relative to the housing, wherein the visibility of the emptying display is affected by moving the view release member.

2. The device according to claim 1, further comprising:
a product container carrying a needle, wherein the container and needle are movable relative to the housing along the longitudinal axis of the housing between an initial position and a piercing position, wherein
the view release member has a first area in the view area when the container and needle are in the initial position and a second area in the view area when the container and needle are in the piercing position, and wherein
the emptying display has a first area in the view area when the product container is in the piercing position, a second area in the view area when the container is empty of product and an intermediate area between the first area and the second area in the view area during the administering of the product.

3. The device according to claim 2, wherein the view release member and the emptying display at least partially overlap.

4. A device for administering an injectable product, comprising:
a) a housing with a housing wall,
b) a look-through element which is disposed on the housing and by which a viewing region can be viewed,
c) a dispensing display movable inside the housing and relative to the housing at least partially through the viewing region, and
d) a view-releasing element movable relative to the housing, wherein the view of the dispensing display can be affected by the view-releasing element.

5. The device as claimed in claim 4, wherein the look-through element is a window.

6. The device as claimed in claim 4, wherein the view-releasing element is between the housing wall and dispensing display.

7. The device as claimed in claim 4, further comprising a product container contained in the device, wherein the product container and a needle connected to the product container can be moved relative to the housing along the longitudinal axis of the housing between an initial position and a piercing position.

8. The device as claimed in claim 7, wherein the product container is coupled with the view-releasing element so that during a movement of one of the view-releasing element or product container the other is at least partly slaved in the movement.

9. The device as claimed in claim 8, wherein the product is dispensed from the product container by a plunger acting on the product, the plunger coupled with the dispensing display so that when one of the dispensing display and plunger moves the other is at least partly slaved in the movement.

10. The device as claimed in claim 7, wherein the view-releasing element comprises a first area in the viewing region when the product container and needle are in the initial position.

11. The device as claimed in claim 10, wherein the first area contains information which can be viewed through the look-through element.

12. The device as claimed in the claim 11, wherein the information comprises at least one of a character, an image and a color.

13. The device as claimed in claim 11, wherein the first area is one of transparent or opaque.

14. The device as claimed in claim 10, further comprising a second area adjacent to the first area, the second area in the viewing region when the product container and needle are in the piercing position.

15. The device as claimed in claim 14, wherein the dispensing display can be read through the look-through area and the second area.

16. The device as claimed in claim 14, wherein the second area one of proximally adjoins the view-releasing element or is a window in the view-releasing element.

17. The device as claimed in claim 16, wherein the second area is made from a transparent material and, if the first area is transparent, the second area is a different color than the first area.

18. The device as claimed in claim 17, wherein the transparent material of the second area is clear or colorless.

19. The device as claimed in claim 18, further comprising an intermediate area between the first area and the second area, the intermediate area at least partially in the viewing region as the product container and needle move from the initial position into the piercing position.

20. The device as claimed in claim 19, wherein the intermediate area comprises a character, an image, a color a material property which indicates that the product container is moving.

21. The device as claimed in claim 7, wherein the dispensing display is coupled with the view-releasing element so that, when the product container and needle are moving from the initial position to the piercing position during a piercing sequence, the dispensing display is slaved to the movement of the view-releasing element.

22. The device as claimed in claim 7, further comprising elastic means comprising a forward driving spring for moving the view-releasing element, product container and the dispensing display in the piercing direction.

23. The device as claimed in claim 7, wherein the dispensing display can be moved in the axial direction relative to the view-releasing element when the product container and needle are in the piercing position or during a product dispensing sequence.

24. The device as claimed in claim 7, further comprising elastic means comprising a dispensing spring for driving at least one of an output element, a plunger rod and a plunger in the dispensing direction.

25. The device e as claimed in claim 7, wherein the dispensing display comprises a first area in the viewing region when the product container is in the piercing position.

26. The device as claimed in claim 25, wherein the first area is moved out of the viewing region during a product dispensing operation.

27. The device as claimed in claim 25, further comprising a second area adjacent to the first area, wherein the second area is in the viewing region and can be viewed when the product has been dispensed from the product containe.

28. The device as claimed in claim 27, wherein the first area and the second area contain information in the form of at least one of a material property, a character, an image and a color, and the information of the first area is different from that of the second area.

29. The device as claimed in claim 28, further comprising an intermediate area between the first area and the second area, the intermediate area in the viewing region or moving through the viewing region during the product dispensing operation.

30. The device as claimed in claim 29, wherein the intermediate area comprises at least one of a character, an image, a color and a material property by which it can be seen that the product container is moving.

31. A method of displaying different modes of a device for administering an injectable product, whereby different information is displayed and can be read through a look-through element, and the information comprises information to the effect that a product container in the device is in an initial position, information to the effect that the product container is in a piercing position and information to the effect that the product contained in the product container has been dispensed.

32. The method as claimed in claim 31, wherein the device comprises a look-through element associated with the housing to create a view area, an emptying display movable inside and relative to the housing at least partially in or through the view area, and a view release member movable relative to the housing, wherein the visibility of the emptying display is affected by moving the view release member.
